(19) United States
(12) Patent Application Publication
(10) Pub. No.: US 2022/0317103 A1
(43) Pub. Date: Oct. 6, 2022

(54) OXY-PYROHYDROLYSIS REACTORS WITH PROTECTED INSERTS

(71) Applicant: 3M INNOVATIVE PROPERTIES COMPANY, St. Paul, MN (US)

(72) Inventor: Phanasouk Vorarath, Cottage Grove, MN (US)

(21) Appl. No.: 17/634,118

(22) PCT Filed: Aug. 6, 2020
(86) PCT No.: PCT/IB2020/057456

§ 371 Date: Feb. 9, 2022

Related U.S. Application Data
(60) Provisional application No. 62/885,043, filed on Aug. 9, 2019.

(51) Int. Cl. G01N 31/12; F23C 3/00; G01N 33/00

(52) U.S. Cl. CPC G01N 31/12; F23C 3/002; G01N 33/0052

(57) ABSTRACT

An oxy-pyrohydrolysis article including a pyrotube and a sample insert are described. The sample insert includes a sample insert tube and a corrosion-resistant protective tube. Methods of conducting oxy-pyrohydrolysis using such articles, including their use for measuring total halogen (e.g., total fluorine) content are also described.

### FIELD

The present disclosure relates to oxy-pyrohydrolysis reactors with protected inserts. Methods for total halogen analysis, in particular, for fluorine content analysis, in various samples using such reactors are also described.

### SUMMARY

Briefly, in one aspect, the present disclosure provides an oxy-pyrohydrolysis reactor comprising: a pyrotube comprising a combustion chamber positioned between a first end and a second end the pyrotube; and a sample insert connected to the first end of the pyrotube. The sample insert comprises a sample insert tube that extends through the first end of the pyrotube such that it is located within the combustion chamber of the pyrotube a first distance X1 from the first end of the pyrotube, and the outer opening is located outside the pyrotube; and a protective tube connected to the sample insert tube extending a second distance X2 from the first end of the pyrotube into the combustion chamber of the pyrotube, wherein the ratio of X2 over X1 is at least 2. The sample insert tube comprises a first material selected from quartz, glass, and combinations thereof; and the protective tube comprises a second material selected from a ceramic, a metal, a metal alloy, and combinations thereof.

In another aspect, the present disclosure provides combustion systems including such oxy-pyrohydrolysis reactors.

In yet another aspect, the present disclosure provides methods using of such oxy-pyrohydrolysis reactors and systems. Such methods comprise delivering, via a sample insert, a sample into a pyrotube from a first end thereof, the sample containing one or more halogen elements; and combusting the sample inside the pyrotube to produce combustion products. In some embodiments, the methods further include analyzing the combustion products to determine the total halogen (e.g., fluorine) content of the sample.

Further details of other aspects and various embodiments of this disclosure are also set forth in the description below. Other features, objects, and advantages of the invention will be apparent from the description and from the claims.

### BRIEF DESCRIPTION OF THE DRAWINGS

FIG. 1 is a schematic diagram of an analytical combustion system.

FIGS. 2A-2C illustrate oxy-pyrohydrolysis reactors.

FIG. 3 illustrates a prior art sample insert tube.

FIG. 4 illustrates an exemplary sample insert tube according to some embodiments of the present disclosure.

FIG. 5 illustrates another exemplary sample insert tube according to some embodiments of the present disclosure.

FIG. 6 illustrates an exemplary oxy-pyrohydrolysis reactor including an exemplary sample insert tube according to some embodiments of the present disclosure.

FIG. 7 illustrates another exemplary oxy-pyrohydrolysis reactor including an exemplary sample insert tube according to some embodiments of the present disclosure.

### DETAILED DESCRIPTION

Commercial analytical combustion systems are available and can be selected to target for halogens. Generally, samples containing halogens are combusted inside a pyrotube placed inside a furnace. Combustion products (e.g., gases) can be analyzed to determine the total content of one or more halogens in the samples. Various analytical techniques for determining element contents in samples are described in, for example, U.S. Pat. Nos. 4,401,763 and 4,285,699.

Improved systems and techniques are disclosed in International Patent Publication WO 2017/172390 A1 (“Oxy-Pyrohydrolysis System and Method for Total Halogen Analysis,” P. Vorarath). Those devices included a combustion-enhancing bed including ceramic fiber or fabrics disposed inside the pyrotube to enhance the combustion and protect the pyrotube from damage by corrosive gases.

Despite the advantages of those devices, the present inventors discovered additional improvements to oxy-pyrohydrolysis systems that may further increase the reliability of total halogen analysis and extend the life of critical components such as the pyrotube.

For example, although the ceramic fibers or fabrics protected portions of the pyrotube previously known to be affected by corrosion, the enhanced combustion and the associated increase in HF concentration introduced new regions of corrosion that may limit the life of such systems. Also, although the systems of WO 2017/172390 A1 could be used with a wide variety of samples, many desirable samples (e.g., blood, soil, and seawater) contain salts, minerals and other contaminants. The presence of these materials in combination with the enhanced combustion can further contribute to increased corrosion. In addition, as the pyrotubes and other components are typically made of quartz, corrosion or etching of these parts by HF or salts can release fluorine from the quartz itself, introducing an error in the measured concentrations. This may not be a significant source of error when high concentrations of halogens are present in the samples themselves but can be a problem when attempting to accurately measure lower concentrations, e.g., less than 500 ppm, less than 250 ppm, or even less than 100 ppm.

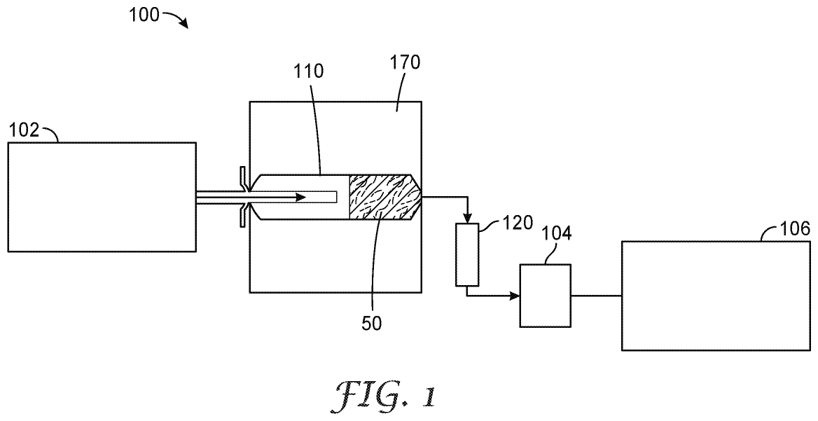

FIG. 1 is a schematic diagram of analytical combustion system 100, for example, a system for analyzing the total halogen content in samples, according to some embodiments of the present disclosure. Test samples (e.g., solids, liquids, emulsions, gases, etc.) can be introduced via sample introduction module 102 into oxy-pyrohydrolysis reactor 110 which can be, for example, a quartz pyrotube. In some embodiments, sample introduction module 102 may include a boat carrier. A known weight of a test portion of a sample can be carried by the boat carrier at a controlled rate into oxy-pyrohydrolysis reactor 110. In some embodiments, sample introduction module 102 may include a liquid delivery instrument such as, for example, a pump, an injector, etc., for continuously delivering liquid samples into the oxy-pyrohydrolysis reactor.

The oxy-pyrohydrolysis reactor is placed inside furnace 170, which operates at high temperatures (e.g., 1000° C. to 1100° C.). Combustion ingredients (e.g., oxygen and/or water) can be provided into oxy-pyrohydrolysis reactor 110 to assist in burning the test samples at high temperatures. Under such oxy-pyrohydrolysis conditions (e.g., water, oxygen, heat, etc.), halogen elements (e.g., chlorine, bromine, fluorine, etc.) contained in the test sample can be converted into combustion products including gaseous compounds such as, for example, fluorides. In some embodiments, the combustion products can be trapped in a condensed steam or buffer inside condenser 120.

An analyzer can be functionally connected to the furnace, e.g., through the condenser, and configured to analyze the composition of the combustion products. In some embodiments, liquid water containing halogen ions (e.g., fluoride ions) can be separated from gases at condenser 120 before it gets transferred, via pump 104, to analyzer 106 where the total content of halogen elements (e.g., fluorine) can be analyzed. Suitable analyzers are known in the field and commercially available including, e.g., a fluoride meter module comprising, e.g., an anion chromatograph or an ion selective electrode. In some embodiments, system 100 can analyze the total fluorine content in a sample that is in the range, e.g., from about 0.005 wt % to about 35 wt %.

In some embodiments, combustion-enhancing bed 50 is provided inside oxy-pyrohydrolysis reactor 110. The combustion-enhancing bed includes ceramic fibers or fabrics which can effectively enhance the combustion of samples inside reactor 110.

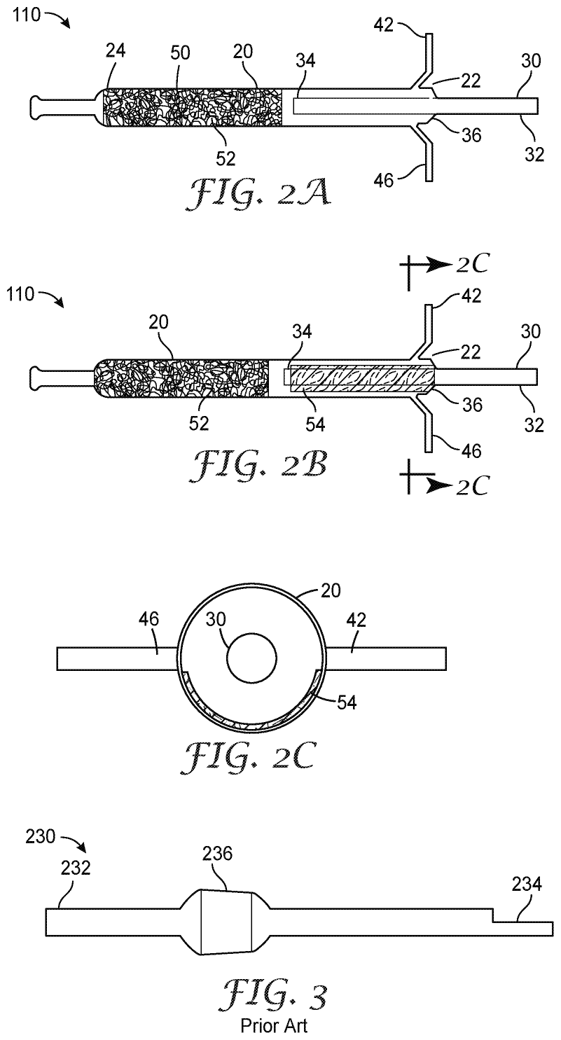

FIGS. 2A-2C illustrate various oxy-pyrohydrolysis reactors 110 and their components, according to various embodiments. Referring to FIG. 2A, oxy-pyrohydrolysis reactor 110 includes pyrotube 20 extending along an axis thereof between first end 22 and second end 24 opposite the first end and defining a combustion chamber. Generally, pyrotube 20 is made of materials that can withstand high temperatures (e.g., about 1100° C. or higher), for example, quartz, glass, ceramics, and metals such as platinum. Typically, the pyrotube is made of quartz or glass, preferably quartz. The dimension of the pyrotube are not critical and may be selected to meet specific needs. In some embodiments, pyrotube 20 can have an internal diameter of, for example, about 10 mm to about 10 cm, and a length of, for example, about 10 cm to about 100 cm.

Fluid inlets 42 and 46 are located near first end 22 of pyrotube 20 and configured to direct combustion ingredients such as, for example, oxygen and water into the body of the pyrotube. It is to be understood that one or more of the combustion ingredients may be optional. For example, for testing liquid samples, additional water may not be needed.

Sample insert 30 extends between first end 32 and second end 34 thereof and is connected to first end 22 of the pyrotube 20 at a junction 36. In some embodiments, the sample insert is a separate component connected to the pyrotube. In some embodiments, the sample insert can be an integral portion of the pyrotube. In some embodiments, the sample insert may have respective desirable structures according to the different type of samples (e.g., solids, liquids, emulsions, gases, etc.) to be delivered. For example, second end 34 may be configured to hold samples comprising a solid, a liquid, an emulsion, or a combination thereof. Alternatively, second end 34 may be configured to deliver fluid samples, e.g., liquids or gases.

In the depicted embodiment of FIG. 2A, optional combustion-enhancing bed 50 is disposed inside pyrotube 20 and downstream of second end 34 of sample insert 30. Combustion-enhancing bed 50 comprises ceramic fiber 52. In some embodiments, ceramic fibers 52 include strings of ceramic fibers that are randomly rolled and packed inside the pyrotube. The packing density of the fibers can be suitable to allow combustion gases and steam vapor to pass through.

In some embodiments, the ceramic fibers can include, for example, alumina-based inorganic oxide fibers. The alumina-based inorganic oxide fibers typically have an average effective fiber diameter of at least about 5 micrometers, although this is not a requirement. In some embodiments, the average effective fiber diameter is less than or equal to 50 micrometers or less than or equal to 25 micrometers.

Useful alumina-based inorganic oxide fibers include, for example, aluminoborosilicate fibers as described in U.S. Pat. No. 3,795,524 (Sowman). In some embodiments, the aluminoborosilicate fibers comprise, on a theoretical oxide basis: about 35 percent by weight to about 75 percent by weight (more preferably, about 55 percent by weight to about 75 percent by weight) of Al2O3; greater than 0 percent by weight (more preferably, at least about 15 percent by weight) and less than about 50 percent by weight (more preferably, less than about 45 percent, and most preferably, less than about 40 percent) of SiO2; and greater than about 1 percent by weight (more preferably, less than about 25 percent by weight, even more preferably, about 1 percent by weight to about 20 percent by weight, and most preferably, about 2 percent by weight to about 15 percent by weight) of B2O3, based on the total weight of the aluminoborosilicate fibers. Preferred aluminoborosilicate fibers are commercially available as NEXTEL 312 inorganic oxide fiber from 3M Company, Maplewood, Minn.

Useful alumina-based inorganic oxide fibers also include aluminosilicate fibers. Aluminosilicate fibers, which are typically crystalline, comprise aluminum oxide in the range from about 67 to about 97 percent by weight and silicon oxide in the range from about 3 to about 33 percent by weight. Aluminosilicate fibers can be made as disclosed, for example, in U.S. Pat. No. 4,047,965 (Karst et al.). In some embodiments, the aluminosilicate fibers include, on a theoretical oxide basis, from about 67 to about 85 percent by weight of Al2O3 and from about 33 to about 15 percent by weight of SiO2, based on the total weight of the aluminosilicate fibers. In some embodiments, the aluminosilicate fibers include, on a theoretical oxide basis, from about 67 to about 77 percent by weight of Al2O3 and from about 23 to about 33 percent by weight of SiO2, based on the total weight of the aluminosilicate fibers. In some embodiments, the aluminosilicate fiber includes, on a theoretical oxide basis, from about 85 to about 97 percent by weight of Al2O3 and from about 3 to about 15 percent by weight of SiO2, based on the total weight of the aluminosilicate fibers. Aluminosilicate fibers are commercially available, for example, as NEXTEL 550 and NEXTEL 720 aluminosilicate fiber from 3M Company.

In some embodiments, the alumina fibers include, on a theoretical oxide basis, greater than about 98 percent by weight of Al2O3 and from about 0.2 to about 1.0 percent by weight of SiO2, based on the total weight of the alumina fibers. Alpha alumina fibers are available, for example, as NEXTEL 610 inorganic oxide fiber from the 3M Company.

Referring now to FIGS. 2B and 2C, in some embodiments, sheet 54 of ceramic fabric or fiber is disposed in pyrotube 20 near first end 22 of the pyrotube. Sheet 54 is positioned between oxygen inlet 42 and water inlet 46. Sheet 54 of ceramic fabric or fiber can help spread and quickly evaporate water from the water inlet along the pyrotube. The porosity of the fabric or fiber can spread the water preventing it from pooling. In some embodiments, sheet 54 can extend, for example, about one-quarter to about one-half of the length of the pyrotube. The ceramic fibers of sheet 54 can be the same or different from ceramic fibers 52 of FIG. 2A.

FIG. 3 is a cross sectional view of sample insert 230 of the prior art. Sample insert 230 extends from first end 232 to second end 234. Sample insert 230 further includes junction 236 configured to allow sample insert 230 to be connected to the first end of a pyrotube. Generally, such pyrotubes are made of quartz or glass to allow easy formation of the structures and to facilitate connection to quartz pyrotubes.

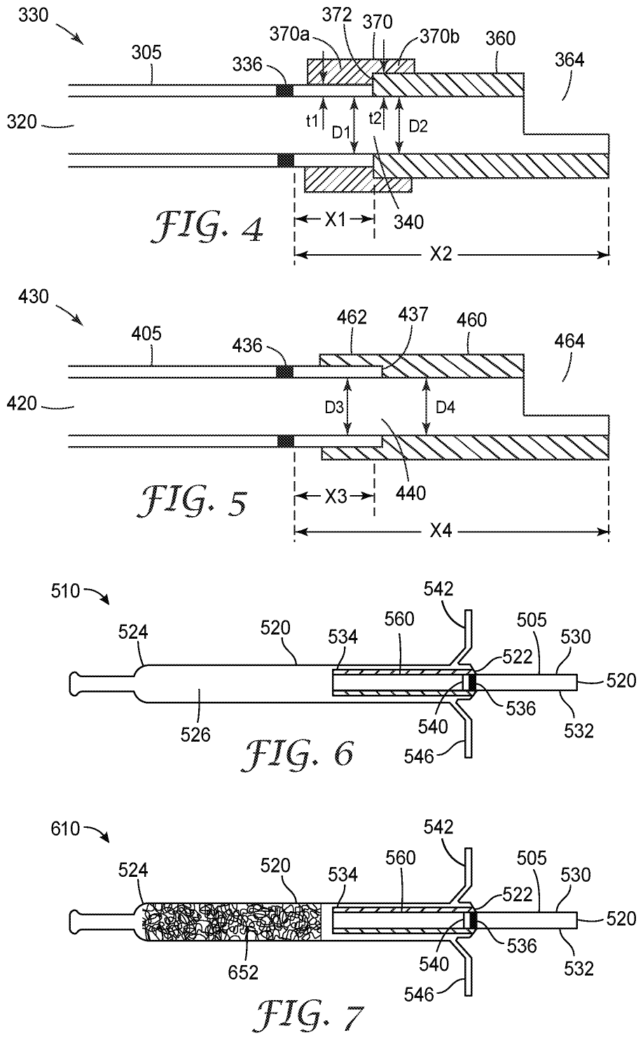

FIG. 4 is a cross sectional view of exemplary sample insert 330, according to some embodiments of the present disclosure. Sample insert 330 can be used in any oxy-pyrohydrolysis reactor including those described herein, and variations thereof. Sample insert 330 includes sample insert tube 305 having inner opening 340 and outer opening 320.

Sample insert 330 further includes protective tube 360 connected to inner opening 340 of sample insert tube 305. Generally, the connection between the protective tube and the inner opening of the sample insert tube is a sealed connection to eliminate leaks. Protective tube 360 includes second end 364, serving as the inner opening of sample insert 330. Second end 364 may be configured to hold samples to be inserted into the pyrotube, e.g., samples comprising solids, liquids, emulsions and combinations thereof. Second end 364 may also be configured to deliver fluid samples, e.g., liquid or gases to the desired location in the pyrotube.

When sample insert 330 is assembled with a pyrotube, the sample insert can be connected to the pyrotube at junction 336, with sample insert tube 305 extending a distance X1 into the pyrotube. In some embodiments, it can be desirable to minimize distance X1 to limit the potential for exposure to the corrosive combustion environment.

When sample insert 330 is assembled with a pyrotube at junction 336, protective tube 360 extends a distance X2 into the combustion chamber of the pyrotube to position second end 364 at the desired location, wherein X2 is greater than X1. In some embodiments, the ratio of X2 over X1 is at least 2, e.g., at 10, at least 20, or even at least 50. In some embodiments, protective tube 360 may cover some of or all of the portion of sample insert tube 305 that extends into the combustion chamber.

Generally, insert tube 305 is made of materials that can withstand high temperatures (e.g., about 1100° C. or higher). Such materials may include, for example, quartz or glass. In some embodiments, insert tube 305 is a quartz tube. In some embodiments, the insert tube may comprise ceramics, metals (e.g., Pt), or metal alloys.

Generally, protective tube 360 is made of materials that can withstand high temperatures, but also resist damage from corrosive gases produced in the combustion process, such as, for example, hydrogen fluoride and/or salts. Without protective tube 360, the portion of insert tube 305 disposed inside a combustion chamber may suffer damage by such corrosive gases. In some embodiments, the protective tubes can withstand high temperatures (e.g., about 1000° C., about 1100° C., or even higher). In some embodiments, protective tube 360 may be made of materials having a melting point of about 1500° C. or higher and may not bend or soften at about 1000° C. to about 1100° C. or higher.

In some embodiments, the protective tube comprises a ceramic. In some embodiments, the protective tube can include one or more metals or metal alloys. In some embodiments, the protective tube can include platinum.

Referring to FIG. 4, in some embodiments, protective tube 360 has an inner diameter D2 substantially the same as an inner diameter D1 of insert tube 305 at inner opening 340 thereof. In the depicted embodiment of FIG. 4, sleeve 370 connects protective tube 360 to insert tube 305. Sleeve 370 has first portion 370a covering the outer surface of insert tube 305 and second portion 370b connected to protective tube 360. In some embodiments, protective tube 360 has a thickness t2 greater than the thickness t1 of inner tube 305 at inner opening 340 thereof. Sleeve 370 may include step 372 between first and second portions 370a and 370b to accommodate the thickness difference of the inner tube and the protective tube.

Sleeve 370 can be connected to inner tube 305 and protective tube 360 by any suitable mechanisms. In some embodiments, sleeve 370 can be connected to the inner tube and the protective tube by, e.g., adhesives to form the sample insert. In some embodiments, the sleeve can be connected to the inner tube and the protective tube by, e.g., a ground joint or a threaded connection. Generally, the connections provide a seal to prevent leakage.

Sleeve 370 can be made of any suitable materials that can resist the corrosion of hydrogen fluoride and/or salts. In some embodiments, sleeve 370 can be a ceramic tube. In some embodiments, the sleeve can include one or more metals or metal alloys (e.g., Pt or its alloy).

FIG. 5 is a cross sectional view of sample insert 430, according to another embodiment. Sample insert 430 can be used in any oxy-pyrohydrolysis reactors described herein or variations thereof. Sample insert 430 includes insert tube 405 having inner opening 440 and outer opening 420. Sample insert 430 includes protective tube 460 connected to inner opening 440 of insert tube 410. When sample insert 430 is assembled with a pyrotube, the sample insert can be connected to the pyrotube at junction 436. When assembled, inner opening 440 of insert tube 405 extends a distance X3 into the combustion chamber, and second end 464 of protective tube 460 extends a distance X4 into the combustion chamber of the pyrotube to form the inner opening of sample insert 430. Generally, X4 is greater than X3. In some embodiments, the ratio of X4 over X3 is at least 2, e.g., at least 5, at least 10 or even at least 20.

In the embodiments, of FIG. 5, protective tube 460 has an inner diameter D4 substantially the same as an inner diameter D3 of insert tube 410 at inner opening 440 thereof. In the depicted embodiment of FIG. 5, protective tube 460 includes sleeve portion 462 connected to an outer surface of inner tube 405. Sleeve portion 462 can be formed by thinning the tube wall of protective tube 460 and making step 437 at the inner surface of protective tube 460. Sleeve portion 462 can be connected to the outer surface of insert tube 410 by any suitable mechanisms such as, for example, tight filing (e.g., pipe to pipe sleeve type fitting), or a threaded or ground joint.

FIG. 6 is a perspective view of an oxy-pyrohydrolysis reactor 510 including a sample insert 530, according to any embodiments of the present disclosure. In some embodiments, sample insert 530 can be, for example, sample insert 330 of FIG. 4. In some embodiments, sample insert 430 of FIG. 5 may be used.

In the depicted embodiment of FIG. 6, pyrotube 520 extends between first end 522 and second end 524 thereof. Pyrotube 520 includes combustion chamber 526, and optional fluid inlets 542 and 546 configured to direct combustion ingredients such as, for example, oxygen and water into the pyrotube.

Sample insert 530 is connected to pyrotube 520 at first end 522 and configured to deliver samples into the combustion chamber 526. Sample insert 530 includes insert tube 505 connected at junction 536. As shown, outer opening 520 of insert tube 505 is positioned outside the pyrotube, while inner opening 540 extends a short distance through first end 522 into pyrotube 520. In some embodiments, insert tube 505 can be a separate part removably sealed to pyrotube 520 at the junction 536.

In some embodiments, insert tube 505 can be made of the same materials of the pyrotubes described herein. For example, both insert tube 505 and pyrotube 520 can be made of quartz or glass, which can be beneficial for forming the junction 536 to connect the parts.

Sample insert 530 further includes protective tube 560 connected to inner tube 505 at the inner opening 540 thereof. Second end 534 of protective tube 560 extends into combustion chamber 526 of the pyrotube to form the inner opening, through which samples can be delivered into the combustion chamber.

FIG. 7 is a perspective view of another exemplary oxy-pyrohydrolysis reactor 610 including the sample insert 530. Oxy-pyrohydrolysis reactor 610 is similar to oxy-pyrohydrolysis reactor 510 of FIG. 6, except oxy-pyrohydrolysis reactor 610 includes combustion-enhancing bed 652 disposed inside pyrotube 520.

Although not shown, in some embodiments, oxy-pyrohydrolysis reactor 510 or 610 may further include a sheet of ceramic fabric disposed inside the pyrotube adjacent to the protective tube. Such a sheet of ceramic fabric can be, for example, sheet 54 of ceramic fabric or fiber disposed on a bottom of the pyrotube 20, as shown in FIG. 2D.

Various modifications and alterations of this invention will become apparent to those skilled in the art without departing from the scope and spirit of this invention.

1. An oxy-pyrohydrolysis reactor comprising:
a pyrotube comprising a combustion chamber positioned between a first end and a second end of the pyrotube; and
a sample insert connected to the first end of the pyrotube, the sample insert comprising:
a sample insert tube having an inner opening and an outer opening, wherein the sample insert tube extends through the first end of the pyrotube such that the inner opening is located within the combustion chamber of the pyrotube a first distance X1 from the first end of the pyrotube, and the outer opening is located outside the pyrotube; and
a protective tube having a first end connected to the inner opening of the sample insert tube and a second end extending a second distance X2 from the first end of the pyrotube into the combustion chamber of the pyrotube, wherein the ratio of X2 over X1 is at least 2;
wherein the sample insert tube comprises a first material selected from quartz, glass, and combinations thereof; and the protective tube comprises a second material selected from a ceramic, a metal, a metal alloy, and combinations thereof.

2. The oxy-pyrohydrolysis reactor of claim 1, wherein the first material comprises quartz and the second material comprises a ceramic.

3. The oxy-pyrohydrolysis reactor of claim 1, wherein the second material comprises one or more metals or metal alloys.

4. The oxy-pyrohydrolysis reactor of claim 3, wherein the second material comprises platinum.

5. The oxy-pyrohydrolysis reactor of claim 1, wherein the ratio of X2 over X1 is at least 20.

6. The oxy-pyrohydrolysis reactor of claim 1, wherein the second end of the protective tube is configured to hold a sample comprising a solid, a liquid, an emulsion, or combinations thereof.

7. The oxy-pyrohydrolysis reactor of claim 1, wherein the second end of the protective tube is configured to deliver a fluid sample comprising a liquid or gas.

8. The oxy-pyrohydrolysis reactor of claim 1, further comprising a sleeve connecting the protective tube to the sample insert tube.

9. The oxy-pyrohydrolysis reactor of claim 1, further comprising a combustion-enhancing bed comprising ceramic fibers or fabrics disposed inside the pyrotube.

10. (canceled)

11. The oxy-pyrohydrolysis reactor of claim 1, wherein the pyrotube further comprises at least one fluid inlet located at the first end of the pyrotube.

12. The oxy-pyrohydrolysis reactor of claim 11, further comprising a sheet of ceramic fabric disposed inside the pyrotube near the at least one fluid inlet.

13. A combustion system comprising a furnace and the oxy-pyrohydrolysis reactor of claim 1, wherein at least a portion of the oxy-pyrohydrolysis reactor is positioned inside the furnace.

14. The combustion system of claim 13, further comprising an analyzer functionally connected to the furnace and configured to analyze a composition of combustion products produced in the pyrotube.

15. The combustion system of claim 14, wherein the analyzer is configured to analyze the total fluorine content in the composition.

16. A method comprising:
delivering, via a sample insert, a sample into a pyrotube from a first end thereof, the sample containing one or more halogen elements, the sample insert connected to the pyrotube at a junction thereof, the sample insert comprising:
a sample insert tube having an inner opening and an outer opening, wherein the sample insert tube extends through the first end of the pyrotube such that the inner opening is located within the combustion chamber of the pyrotube a first distance X1 from the first end of the pyrotube, and the outer opening is located outside the pyrotube; and
a protective tube having a first end connected to the inner opening of the sample insert tube and a second end extending a second distance X2 into the combustion chamber of the pyrotube, wherein the ratio of X2 over X1 is at least 2;
wherein the sample insert tube comprises a first material selected from quartz, glass, and combinations thereof; and the protective tube comprises a second material selected from a ceramic, a metal, a metal alloy, and combinations thereof; and
combusting the sample inside the pyrotube to produce combustion products.

17. The method of claim 16 further comprising analyzing the combustion products to determine a total halogen content in the sample.

18. The method of claim 16, wherein the first material comprises quartz and the second material comprises a ceramic.

19. The method of claim 16, further comprising a combustion-enhancing bed disposed inside the pyrotube adjacent to a second end of the pyrotube, the combustion-enhancing bed comprising ceramic fibers or fabrics.

20. (canceled)

21. The method of claim 16, wherein the combustion products comprise HF.

22. The method of claim 16, wherein the sample comprises at least one salt.

23. (canceled)
